(19) United States
(12) Patent Application Publication
(10) Pub. No.: US 2018/0296081 A1
(43) Pub. Date: Oct. 18, 2018

(54) WIRELESS LARYNGOSCOPE

(71) Applicants: Thomas L. Cook, Naples, FL (US); Michael T. Williams, Winston-Salem, NC (US)

(72) Inventors: Thomas L. Cook, Naples, FL (US); Michael T. Williams, Winston-Salem, NC (US)

(21) Appl. No.: 15/490,732

(22) Filed: Apr. 18, 2017

(51) Int. Cl. A61B 1/267; A61B 1/04; A61B 1/06; A61B 1/00

(52) U.S. Cl. CPC A61B 1/267; A61B 1/04; A61B 1/00032; A61B 1/00016; A61B 1/06

(57) ABSTRACT

Particular embodiments of the invention comprise a wireless laryngoscope and a blade forming a portion of a wireless laryngoscope. The wireless laryngoscope comprises a handle having a front and rear portion, a blade having an outer surface and an inner surface, a blade tip having a convex outer surface and a concave inner surface, and a blade bend located at an end of the blade opposite the blade tip.

### BACKGROUND OF THE INVENTION

### Field of the Invention

The present invention relates to a wireless laryngoscope and, in particular, a wireless laryngoscope with an improved blade tip.

### Description of the Related Art

A laryngoscope is often used in a variety of situations such as surgical and emergency procedures. While a patient may be conscious during one or more of these procedures, the patient may also be unconscious and unable to actively assist the individual using the laryngoscope. If the patient is unconscious, he or she will most likely not have control of his or her bodily movements, so the patient would be unable to assist the individual using the laryngoscope by positioning his or her tongue in a proper manner or opening his or her mouth wide enough and in such a manner as to minimize, or altogether prevent, dental damage caused by contact with the laryngoscope. In situations such as this, it is often difficult for an individual to effectively use the laryngoscope, which may jeopardize a patient's life if the purpose for the use is to identify and remove foreign objects from the throat. Therefore, there is a need for an improved wireless laryngoscope that addresses these problems.

### SUMMARY OF THE INVENTION

Particular embodiments of the invention comprise a wireless laryngoscope and a blade forming a portion of a wireless laryngoscope. Certain embodiments of the wireless laryngoscope comprise a handle having a front portion and a rear portion. The front portion of the handle is arranged opposite from the rear portion of the handle. The wireless laryngoscope further comprises a blade having an outer surface and an inner surface. The outer surface of the blade is arranged opposite from the inner surface. The outer surface of the blade is also arranged on a same side of the wireless laryngoscope as the front portion of the handle, and the inner surface of the blade is arranged on a same side of the wireless laryngoscope as the rear portion of the handle. The wireless laryngoscope further comprises a blade tip, which has a convex outer surface corresponding to the outer surface of the blade and a concave inner surface corresponding to the inner surface of the blade. In addition, the wireless laryngoscope comprises a blade bend located between the handle and the blade, and the blade bend is located at an end of the blade opposite the blade tip.

Certain embodiments of the blade forming a portion of the wireless laryngoscope comprise an outer surface and an inner surface, wherein the outer surface of the blade is arranged opposite from the inner surface. The blade further comprises a blade tip having a convex outer surface corresponding to the outer surface of the blade and a concave inner surface corresponding to the inner surface of the blade. In addition, the blade comprises a blade bend, which is located at an end of the blade opposite the blade tip. The blade also comprises a light source located on the inner surface of the blade, wherein an angle formed between the light source and the blade tip is twenty degrees.

The foregoing and other embodiments, objects, features, and advantages of the invention will be apparent from the following more detailed descriptions of particular embodiments of the invention, as illustrated in the accompanying drawings wherein like reference numbers represent like parts of the invention.

### BRIEF DESCRIPTION OF THE FIGURES

FIG. 1 is a left side view of a wireless laryngoscope, in accordance with an embodiment of the invention.

FIG. 2 is a front view of the wireless laryngoscope of FIG. 1.

FIG. 3 is a right side view of the wireless laryngoscope of FIG. 1.

FIG. 4 is a rear view of the wireless laryngoscope of FIG. 1.

FIG. 5 is a bottom view of the wireless laryngoscope of FIG. 1 taken along the line 5-5 in FIG. 3.

FIG. 6 is a close-up view of a portion of the bottom of the wireless laryngoscope of FIG. 1 taken within the circle labeled FIG. 6 in FIG. 5.

FIG. 7 is a cutaway view of the wireless laryngoscope of FIG. 1 taken along the line 7-7 in FIG. 3.

FIG. 8 is a cutaway view of the wireless laryngoscope of FIG. 1 taken along the line 8-8 in FIG. 4.

FIG. 9 is an upper perspective view of the wireless laryngoscope of FIG. 1.

FIG. 10 is a lower perspective view of the wireless laryngoscope of FIG. 1.

FIG. 11 is a side view of a wireless laryngoscope in communication with a Wi-Fi enabled device, in accordance with an embodiment of the invention.

FIG. 12 is a side view of a wireless laryngoscope, in accordance with an embodiment of the invention.

FIG. 13 is an upper perspective view of a wireless laryngoscope, in accordance with an embodiment of the invention.

### DETAILED DESCRIPTION OF THE INVENTION

Embodiments of the invention comprise a wireless laryngoscope and a blade forming a portion of a wireless laryngoscope. Particular embodiments are further described herein, in association with the figures included herewith.

In particular embodiments, the invention comprises a wireless laryngoscope. The wireless laryngoscope has a handle, and the handle has a front portion and a rear portion. The rear portion of the handle is arranged opposite from the front portion of the handle. In the conventional use of the wireless laryngoscope, the front portion of the handle is the side of the handle which will be closest to a patient's teeth, and the rear portion of the handle is the side of the handle which will be furthest from a patient's teeth.

The handle may be textured such that a user may more easily grip one or more projections (e.g., ridges) or indentations (e.g., grooves) when using the wireless laryngoscope. The handle may also be made of a variety of materials. In particular embodiments, the handle is made of a plastic material. Further, the handle has a thickness, which is the linear distance from the outermost portion of the front portion of the handle to the outermost portion of the rear portion of the handle. In particular embodiments, the handle of the wireless laryngoscope is cylindrical, and the thickness of the handle is the diameter taken along a cross section of a cylinder.

The wireless laryngoscope further comprises a blade, and the blade has an outer surface and an inner surface. The outer surface of the blade is arranged opposite from the inner surface of the blade such that the outer surface of the blade is arranged on the same side of the wireless laryngoscope as the front portion of the handle, and the inner surface of the blade is arranged on the same side of the wireless laryngoscope as the rear portion of the handle.

Together with the inner surface, the outer surface of the blade may form an angle. In particular embodiments, the outer surface of the blade is a convex surface, and the inner surface of the blade is a concave surface. In such embodiments, the convex (i.e., contoured) outer surface provides greater comfort to a patient when the wireless laryngoscope is used by an individual. In addition, while the blade may be made from materials such as stainless steel, the blade is made from plastic in preferred embodiments. In such embodiments, the blade may be manufactured more inexpensively, may be more easily sterilized, and is disposable. Further, in the embodiments in which the blade is made from plastic, the blade may be made from a softer plastic than the plastic used to form the handle of the wireless laryngoscope. With respect to such embodiments, the softer plastic used to form the blade minimizes, or altogether removes, the risk of a patient experiencing dental damage.

In particular embodiments, the blade further comprises a camera, which is located on the inner surface of the blade. In particular embodiments, the camera includes a transmitter, and the transmitter is capable of connecting to a device via radio frequency (“RF”) waves. In particular embodiments, the device is a Wi-Fi enabled device, although any means of RF communication (e.g., Bluetooth) are also contemplated herein. In such embodiments, the camera can link to and display the view of the camera, which is substantially in the direction of the blade away from the handle, on any other Wi-Fi enabled device, such as a smart phone, laptop, tablet, desktop, smart television, or any other device capable of connecting to the Internet via a Wi-Fi connection. The capability of the transmitter to connect to a Wi-Fi enabled device eliminates hardwired communication with a separate device, which requires cords and can pose a danger to both the individual operating the laryngoscope as well as the patient. In further embodiments, the camera is powered by one or more batteries, which are connected to a circuit board comprised of a Wi-Fi integrated circuit, a driver for a light source, and an input for the camera (all of which are powered by the battery) and allow the camera to be operated in a remote location away from a standard electrical outlet. In such embodiments, the existence of the one or more batteries allows an individual using the wireless laryngoscope to utilize the instrument in locations such as emergency rooms, ambulatory situations, and field use (e.g., military base camps or operations).

In such embodiments in which the transmitter is able to connect to the Internet via a Wi-Fi connection, an application for the use on a Wi-Fi enabled medical device is utilized. The application connects the wireless laryngoscope to the medical device via the transmitter and Wi-Fi connection in order to allow an individual using the wireless laryngoscope to stream video to the medical device, which, in particular embodiments, is the end-user's medical device. The video stream enables the end-user to observe the placement and insertion of an endotracheal tube into the trachea, whether placed orally or nasally. In addition, an end-user may also observe the placement and insertion of a transesophageal echocardiography (“TEE”) probe, a nasogastric or orogastric tube, esophageal temperature probe, and/or a nasal airway. Through the use of the application, an end-user may record video onto the end-user's device, which may be used to demonstrate successful placement of medical devices, the absence or presence of trauma after the procedure, and to teach other users of the wireless laryngoscope of the proper use thereof.

In particular embodiments, the blade of the wireless laryngoscope further comprises a light source, which is located on the inner surface of the blade. The light source may be located either in the same location as the camera or in a different location than the camera. Much like the camera, the light source is positioned in a manner to provide illumination aimed towards the direction of the blade away from the handle. In further embodiments, the light source is powered by one or more batteries, which allow the light source to be operated in a remote location away from a standard electrical outlet. As is the case with the camera, in such embodiments, the existence of one or more batteries allows an individual using the wireless laryngoscope to utilize the instrument in locations such as emergency rooms, ambulatory situations, and field use (e.g., military base camps or operations).

In particular embodiments, the blade may be manufactured to be a variety of sizes. In this way, the blade of the wireless laryngoscope may be utilized in order to be most useful for specific patients based upon weight, age, and the presence of one or more foreign objects within a patient's throat.

The blade of the wireless laryngoscope also comprises a blade tip. The blade tip has a convex outer surface corresponding to the outer surface of the blade and a concave inner surface corresponding to the inner surface of the blade. In this manner, the blade tip may be referred to as “spoon-shaped,” which, during use, provides better lift of a patient's tongue. In embodiments in which the blade includes a camera and/or a light source, the “spoon-shaped” blade tip assists in providing a more clear view into a patient's esophagus for the camera and/or light source. In such embodiments, the blade tip of the present invention also prevents a patient's tongue from falling off of one or more sides of the blade, which would obscure the view of the camera and/or light source. The view of the camera and/or light source is not the same as the angle of viewing needed for direct viewing by a physician or other individual operating the wireless laryngoscope. In still further embodiments, the blade tip may be both “spoon-shaped” and “T-shaped,” which provides further support for a patient's tongue during the use of the wireless laryngoscope.

In particular embodiments, much like the blade, the blade tip is made from plastic. Further, in the embodiments in which the blade tip is made from plastic, the blade tip may be made from the same plastic as the handle and blade or may be made from a softer plastic than the plastic used to form the handle and/or blade of the wireless laryngoscope. With respect to such embodiments, the softer plastic used to form the blade tip minimizes, or altogether removes, the risk of a patient experiencing dental damage. In further embodiments, the blade tip may be manufactured to be a variety of sizes. In this way, the blade tip of the wireless laryngoscope may be utilized in order to be most useful for specific patients based upon weight, age, and the presence of one or more foreign objects within a patient's throat.

In embodiments in which the wireless laryngoscope further comprises a light source, an angle is formed between the light source and the blade tip. In such embodiments, the angle between the light source and the blade tip is approximately twenty degrees. When the angle between the light source and the blade tip is approximately twenty degrees, this angle enables an easier and more successful intubation of a more difficult airway, such as an airway that is obstructed by a foreign object.

In addition, the wireless laryngoscope further comprises a blade bend. The blade bend is located between the handle and the blade at an end of the blade opposite the blade tip, and the blade bend forms a junction between the front portion of the handle and the outer surface of the blade. In particular embodiments, the handle, blade, blade tip, and blade bend are formed (i.e., molded or manufactured) together as single, unitary structure.

In particular embodiments, much like the blade and blade tip, the blade bend is made from plastic. Further, in the embodiments in which the blade bend is made from plastic, the blade bend may be made from the same plastic as the handle, blade, and blade tip or may be made from a softer plastic than the plastic used to form the handle, blade, and/or blade tip of the wireless laryngoscope. With respect to such embodiments, the softer plastic used to form the blade bend minimizes, or altogether removes, the risk of a patient experiencing dental damage. In further embodiments, the blade bend may be manufactured to be a variety of sizes. In this way, the blade bend of the wireless laryngoscope may be utilized in order to be most useful for specific patients based upon weight, age, and the presence of one or more foreign objects within a patient's throat.

Further, the blade bend has a thickness, which is the linear distance from the outermost portion of the portion of the blade bend adjoining the front portion of the handle to the outermost portion of the portion of the blade bend adjoining the rear portion of the handle. In particular embodiments, the thickness of the blade bend is less than the thickness of the handle. As the blade bend is the area where a patient's front teeth will often close upon and contact the laryngoscope, the thinner blade bend simultaneously allows for easier insertion into the throat of an obese patient while also minimizing, or altogether removing, the risk of a patient experiencing dental damage.

Particular embodiments of the wireless laryngoscope and the blade forming a portion of a wireless laryngoscope discussed above will now be described in further detail below in association with the figures filed herewith exemplifying the present invention in association with particular embodiments of wireless laryngoscopes.

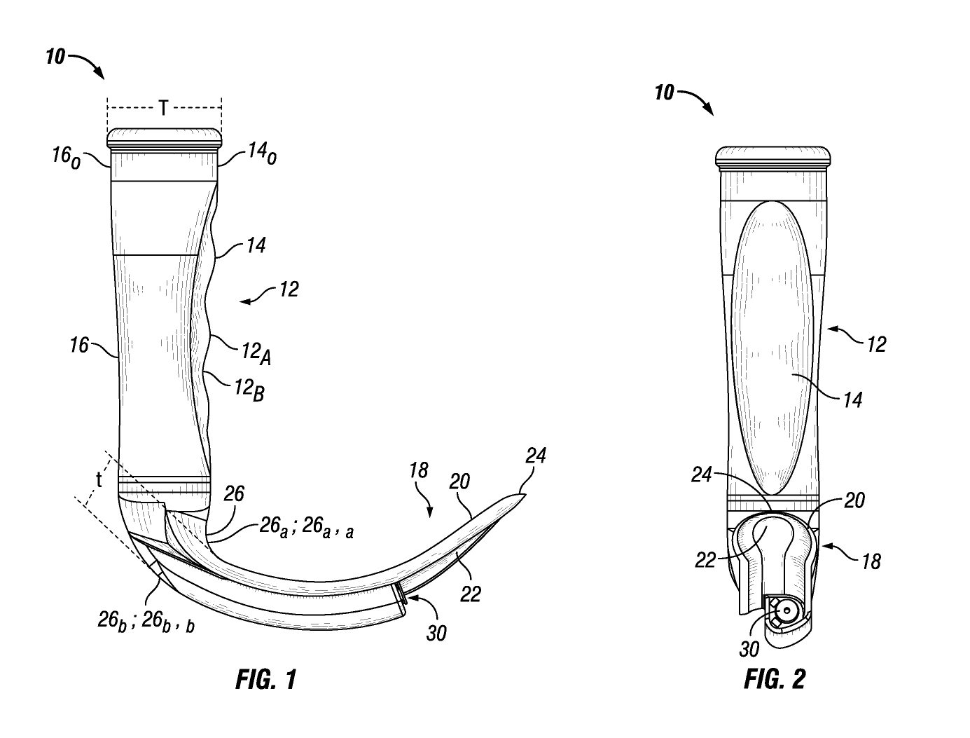

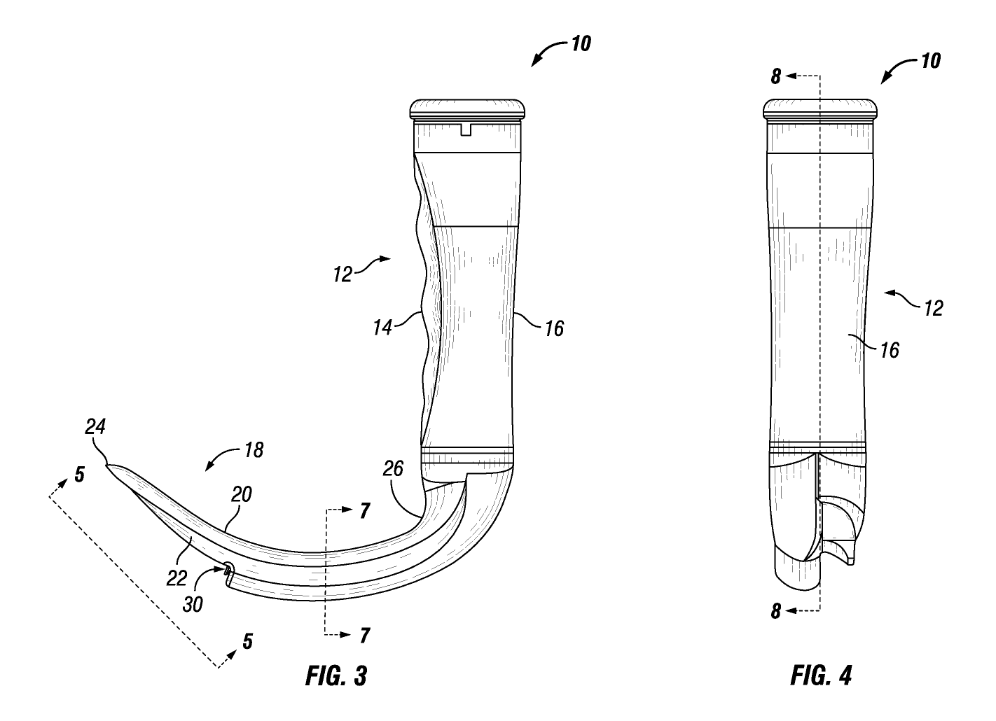

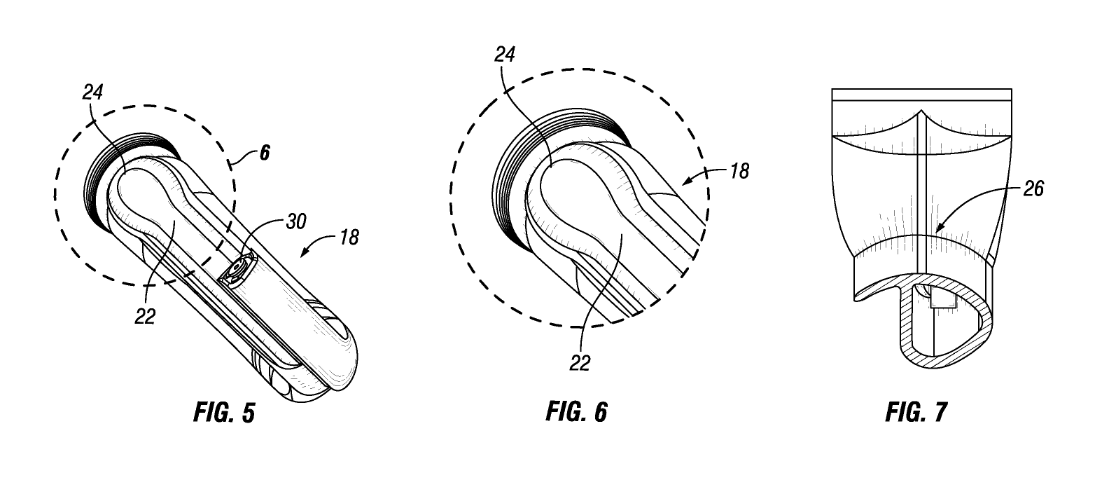

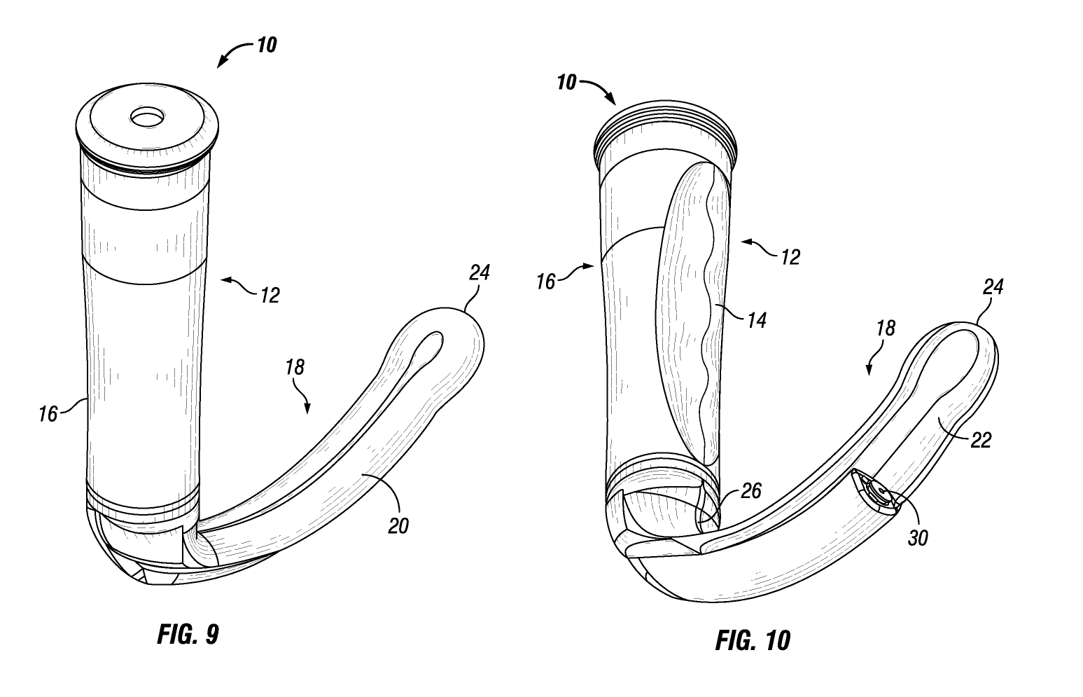

With reference to an exemplary embodiment shown in FIGS. 1-10, a wireless laryngoscope 10 comprises a handle 12, and the handle 12 has a front portion 14 and a rear portion 16. The rear portion 16 of the handle 12 is arranged opposite from the front portion 14 of the handle 12. As shown in the exemplary embodiment in FIGS. 1-10, the handle 12 is textured such that a user may more easily grip one or more projections 12_{A} or indentations 12_{B} when using the wireless laryngoscope 10. Further, the handle 12 has a thickness T, which is the linear distance from the outermost portion 14_{O} of the front portion 14 of the handle 12 to the outermost portion 16_{O} of the rear portion 16 of the handle 12.

The wireless laryngoscope 10 further comprises a blade 18, and the blade 18 has an outer surface 20 and an inner surface 22. As illustrated in FIG. 1, the outer surface 20 of the blade 18 is arranged opposite from the inner surface 22 of the blade 18. In this way, the outer surface 20 of the blade 18 is arranged on the same side of the wireless laryngoscope 10 as the front portion 14 of the handle 12, and the inner surface 22 of the of the blade 18 is arranged on the same side of the wireless laryngoscope 10 as the rear portion 16 of the handle 12. Together with the inner surface 22 of the blade 18, the outer surface 20 of the blade 18 may form an angle. In particular embodiments, and especially as illustrated in FIGS. 2, 9, and 10, the outer surface 20 of the blade 18 is a convex surface, and the inner surface 22 of the blade 18 is a concave surface.

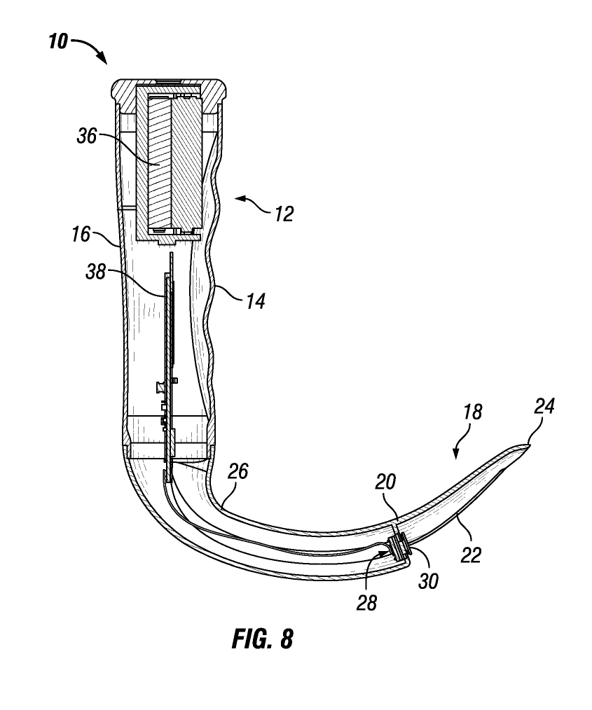

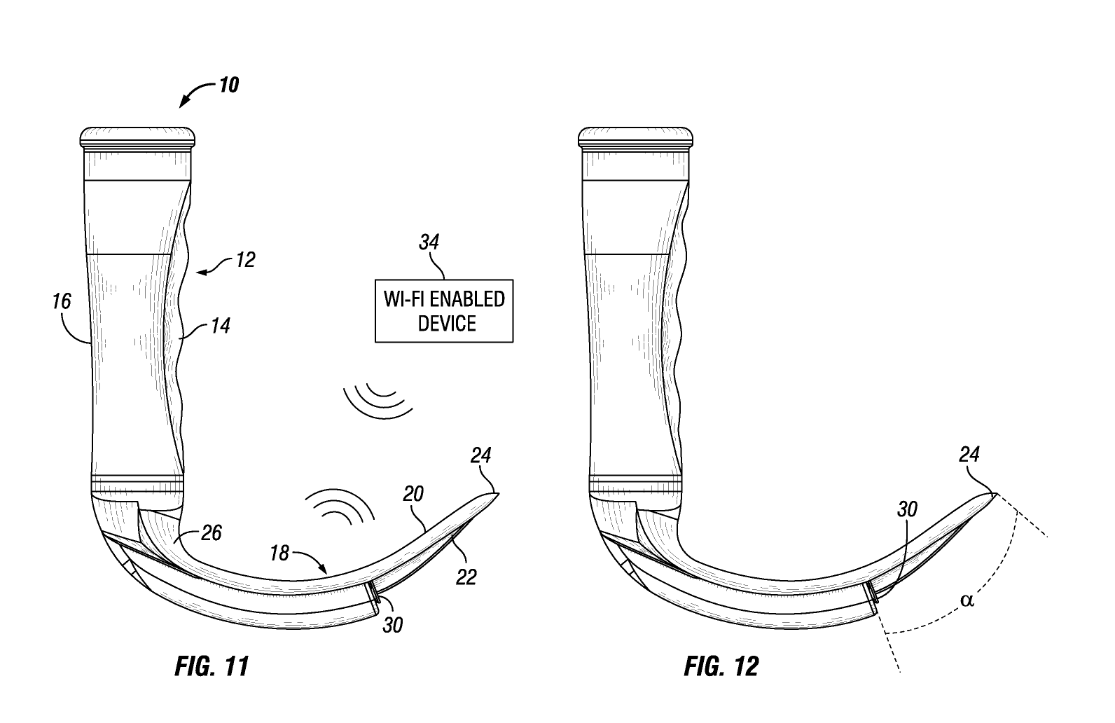

Optionally, and as shown in FIG. 8, the blade 18 further comprises a camera 28, which is located on the inner surface 22 of the blade 18. In particular embodiments, such as the embodiment shown in FIG. 11, the camera 28 includes a transmitter, and the transmitter is capable of connecting to a Wi-Fi enabled device 34. In such embodiments, the camera 28 can link to and display the view of the camera 28, which is substantially in the direction of the blade 18 away from the handle 12, on any other Wi-Fi enabled device 34. In further embodiments, the camera 28 is powered by one or more batteries 36, which are connected to a circuit board 38 and allow the camera 28 to be operated in a remote location away from a standard electrical outlet.

In particular embodiments, such as those illustrated in FIGS. 1-3, 5, 8, and 10-12, the blade 18 of the wireless laryngoscope 10 further comprises a light source 30, which is located on the inner surface 22 of the blade 18. The light source 30 may be located either in the same location as the camera 28 (as shown in FIG. 8) or in a different location than the camera 28. Much like the camera 28, the light source 30 is positioned in a manner to provide illumination aimed towards the direction of the blade 18 away from the handle 12. In some embodiments, the light source 30 is powered by one or more batteries 36, which allow the light source 30 to be operated in a remote location away from a standard electrical outlet.

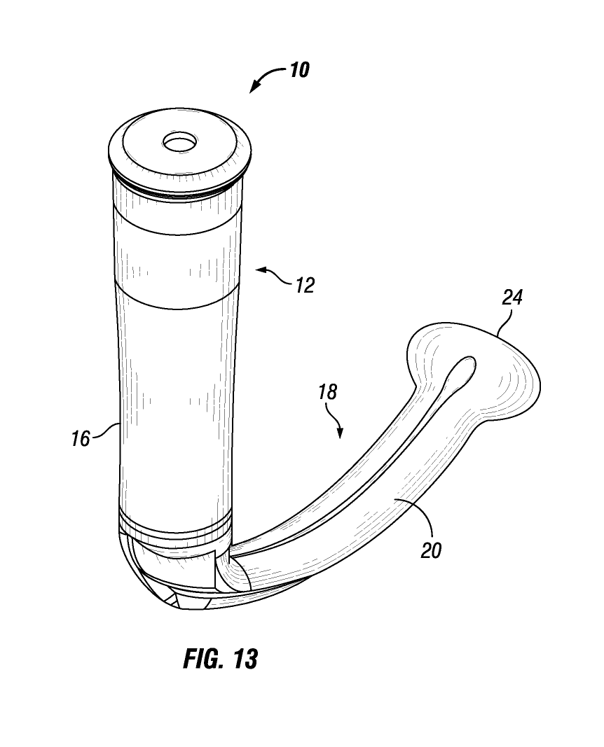

The blade 18 of the wireless laryngoscope 10 also comprises a blade tip 24. The blade tip 24 has a convex outer surface corresponding to the outer surface 20 of the blade 18 and a concave inner surface corresponding to the inner surface 22 of the blade. In this manner, the blade tip may be referred to as “spoon-shaped,” which, during use of the wireless laryngoscope 10 provides better lift of a patient's tongue. In further embodiments, and as illustrated in FIG. 13, the blade tip 24 may be both “spoon-shaped” and “T-shaped,” which provides further support for a patient's tongue during the use of the wireless laryngoscope 10. In embodiments in which the wireless laryngoscope 10 further comprises a light source 30, as illustrated in exemplary embodiments in FIGS. 1-3, 5, 8, and 10-12, an angle α, as illustrated in FIG. 12 is formed between the light source 30 and the blade tip 24. In such embodiments, the angle α between the light source 30 and the blade tip 24 is approximately twenty degrees.

In exemplary embodiments, the wireless laryngoscope further comprises a blade bend 26, as illustrated in FIGS. 1, 3, 7, 8, 10, and 11. The blade bend 26 is located between the handle 12 and the blade 18 at an end of the blade 18 opposite the blade tip 24, and the blade bend 26 forms a junction between the front portion 14 of the handle 12 and the outer surface 20 of the blade 18. In particular embodiments, the handle 12, blade 18, blade tip 24, and blade bend 26 are formed (i.e., molded or manufactured) together as a single, unitary structure comprising the wireless laryngoscope 10.

Further, the blade bend 26 has a thickness t, which is the linear distance from the outermost portion 26_{o,a} of the portion 26ₐ of the blade bend 26 adjoining the front portion 14 of the handle 12 to the outermost portion 26_{o,b} of the portion 26_{b} of the blade bend 26 adjoining the rear portion 16 of the handle 12. In particular embodiments, the thickness t of the blade bend 26 is less than the thickness T of the handle 12.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The terms “at least one” and “one or more” are used interchangeably. The term “single” shall be used to indicate that one and only one of something is intended. Similarly, other specific integer values, such as “two,” are used when a specific number of things is intended. The terms “preferably,” “preferred,” “prefer,” “optionally,” “may,” and similar terms are used to indicate that an item, condition or step being referred to is an optional (i.e., not required) feature of the invention. Ranges that are described as being “between a and b” are inclusive of the values for “a” and “b” unless otherwise specified.

While this invention has been described with reference to particular embodiments thereof, it shall be understood that such description is by way of illustration only and should not be construed as limiting the scope of the claimed invention. Accordingly, the scope and content of the invention are to be defined only by the terms of the following claims. Furthermore, it is understood that the features of any specific embodiment discussed herein may be combined with one or more features of any one or more embodiments otherwise discussed or contemplated herein unless otherwise stated.

1. A wireless laryngoscope comprising:
a handle having a front portion and a rear portion, wherein the front portion is arranged opposite from the rear portion;
a blade having an outer surface and an inner surface, wherein the outer surface of the blade is arranged opposite from the inner surface, further wherein the outer surface of the blade is arranged on a same side of the wireless laryngoscope as the front portion of the handle and the inner surface of the blade is arranged on a same side of the wireless laryngoscope as the rear portion of the handle;
wherein the blade has a blade tip, the blade tip having a convex outer surface corresponding to the outer surface of the blade and a concave inner surface corresponding to the inner surface of the blade; and
a blade bend located between the handle and the blade, the blade bend being located at an end of the blade opposite the blade tip.

2. The wireless laryngoscope of claim 1, wherein the outer surface of the blade is a convex surface and the inner surface of the blade is a concave surface.

3. The wireless laryngoscope of claim 1, wherein the handle, blade, blade tip, and blade bend are formed together as a single structure.

4. The wireless laryngoscope of claim 3, wherein the handle, blade, blade tip, and blade bend are formed of a plastic material.

5. The wireless laryngoscope of claim 1, wherein a thickness of the blade bend is thinner than a thickness of the handle.

6. The wireless laryngoscope of claim 4, wherein the blade bend is made of a softer plastic than the blade and the blade tip.

7. The wireless laryngoscope of claim 4, wherein the blade tip is made of a softer plastic than the handle.

8. The wireless laryngoscope of claim 4, wherein the blade is made of a softer plastic than the handle.

9. The wireless laryngoscope of claim 4, where each of the blade bend, the blade tip, and the blade are made of a softer plastic than the handle.

10. The wireless laryngoscope of claim 1, further comprising:
a camera and a light source, wherein each of the camera and the light source are located on the inner surface of the blade.

11. The wireless laryngoscope of claim 10, wherein the camera and the light source are powered by one or more batteries.

12. The wireless laryngoscope of claim 10, wherein an angle formed between the light source and the blade tip is twenty degrees.

13. The wireless laryngoscope of claim 10, wherein the camera includes a transmitter, wherein the transmitter is capable of connecting to a Wi-Fi enabled device.

14. The wireless laryngoscope of claim 1, wherein the blade tip is T-shaped.

15. A blade forming a portion of a wireless laryngoscope comprising:
an outer surface and an inner surface, wherein the outer surface of the blade is arranged opposite from the inner surface;
a blade tip having a convex outer surface corresponding to the outer surface of the blade and a concave inner surface corresponding to the inner surface of the blade;
a blade bend located at an end of the blade opposite the blade tip; and
a light source located on the inner surface of the blade, wherein an angle formed between the light source and the blade tip is twenty degrees.

16. The blade of claim 15, wherein each of the blade, blade tip, and blade bend are made of plastic.

17. The blade of claim 16, wherein the blade tip is made of a softer plastic than the blade.

18. The blade of claim 16, wherein the blade bend is made of a softer plastic than the blade.

19. The blade of claim 16, wherein each of the blade tip and the blade bend are made of a softer plastic than the blade.

20. The blade of claim 15, wherein the blade tip is T-shaped.
